(19) United States
(12) Patent Application Publication
(10) Pub. No.: US 2026/0144461 A1
(43) Pub. Date: May 28, 2026

(54) BLOOD COMPONENT ASSAYER

(71) Applicant: DYMSENSE LTD., Rishon LeTsiyon (IL)

(72) Inventors: Dor Heller Zarbel, Tel Aviv (IL); Rahav Raviv, Gedera (IL); Eyal Meron, Rosh Ha'Ayin (IL)

(21) Appl. No.: 19/119,777

(22) PCT Filed: Oct. 23, 2023
(86) PCT No.: PCT/IL2023/051094

§ 371 Date: Apr. 10, 2025

Related U.S. Application Data
(60) Provisional application No. 63/418,568, filed on Oct. 23, 2022.

(51) Int. Cl. A61B 5/1455; A61B 5/00; A61B 5/145

(52) U.S. Cl. CPC A61B 5/1455; A61B 5/0059; A61B 5/14546; A61B 5/4845; A61B 5/6826

(57) ABSTRACT

Apparatus for assaying a blood component of a person, that assays a person's blood component responsive to illumination of a distal phalangeal joint (DIP!) of the person's finger.

### RELATED APPLICATIONS

The present application claims the benefit under 35 U.S.C. 119(e) of U.S. Provisional Application 63/418,568 filed on Oct. 23, 2022, the disclosure of which is incorporated herein by reference.

### FIELD

Embodiments of the disclosure relate to methods and systems for assaying a blood component of a person.

### BACKGROUND

Substance abuse is a major contributing factor in causing road accidents. In particular, alcohol abuse, because of the ease with which alcohol may be acquired, its popular acceptance, and frequent consumption by most segments of the population, contributes significantly to traffic accidents and is a contributing factor to almost a third of all traffic deaths in the US. For example, in 2020 in the U.S. 11,654 people died in drunk driving crashes—about one person every 45 minutes! And whereas it is commonly assumed that relatively low levels of blood alcohol concentration (BAC) that might for example result from social drinking do not significantly impair a driver's driving performance, even low levels of BAC have been found to reduce driving competence. BACs in a range from about 0.01 to about 0.07 grams per deciliter (g/L) were indicated as a contributing factor in the traffic deaths of over 2,000 people in 2020.

Accurate and easy to use methods and apparatus for measuring blood components of person that might affect the person's competence to carry out a task would be advantageous. Technology for rapidly and accurately determining a person's BAC prior to allowing the person to take the driving wheel of a vehicle would appear to be particularly advantageous for controlling substance abuse related traffic accidents.

### SUMMARY

An aspect of an embodiment of the disclosure relates to providing methods and systems for relatively quickly and relatively accurately assaying a person's blood component by measuring absorption of light in an assay region of the finger. Optionally, the assay region is a volar region of a distal interphalangeal joint (DIPJ) of a finger.

In an embodiment, the assay is performed by a relatively rapid and accurate assayer (RACAS) that illuminates the DIPJ volar region of the person's finger with light, also referred to as assaying light, in a bandwidth for which the blood component has a relatively large coefficient of absorption and measures intensity of the illuminating light that subsequently emerges from the region. RACAS may illuminate substantially the same volar region with light, hereinafter also referred to as calibrating light, in a bandwidth for which the blood component has a relatively small or substantially zero coefficient of absorption, and measures intensity of the calibrating light which subsequently emerges from the region. RACAS processes the measurements of the emergent assaying and calibration light to provide an assay of the blood component.

In accordance with an embodiment of the disclosure RACAS comprises a controller and a finger cradle into or onto which a person to have a blood component assayed places a finger. The cradle comprises at least one light source operable to transmit assaying and/or calibration light at respective known intensities to illuminate a region of a finger placed in or on the cradle and at least one light sensor for receiving and providing a measurement of intensity of light, emergent light, from the transmitted assaying and/or calibration light that emerges from the illuminated region of the finger. Optionally, the RACAS cradle comprises a fingerprint detector and performs a blood component assay after RACAS determines that the person has properly positioned a finger on the fingerprint detector so that the RACAS controller may image and optionally identify a fingerprint of the finger.

In an embodiment, the at least one light source and at least one light sensor are positioned at fixed locations relative to the fingerprint reader so that when a person's, optionally index, finger is properly placed on the reader, assaying and/or calibration light transmitted by the at least one source illuminates the DIPJ assay region and the at least one light sensor receives emergent assaying and/or calibration light. The at least one light source and at least one light sensor may be mounted on motion stages. When a person places a finger in or on the RACAS cradle, the controller operates the motion stages to align the at least one light source and at least one light sensor with the finger DIPJ assay region so that RACAS may assay the blood component.

An aspect of an embodiment of the disclosure relates to providing a “canister detector” comprising the at least one light source and the at least one light sensor for respectively transmitting and sensing assaying and/or calibration light. Optionally, the at least one light source and at least one light sensor are housed in or on a same can of the canister detector. In an embodiment the at least one light source comprises a plurality of light sources that are configured in an annular array on an outer rim of the can and are oriented to transmit light to a substantially same focal region. The at least one light sensor may be located inside the can at a distance from the rim so that the can wall functions to reduce ambient light from reaching the at least one sensor.

In an embodiment the blood component is alcohol and RACAS operates to provide a measure of BAC. To provide the BAC, RACAS may radiate assaying light in a bandwidth between about 2,000 nanometers (nm) and 2500 nm having a central wavelength of about 2350 nm. RACAS radiates calibration light that is not absorbed by ethanol in a bandwidth between about 1100 and 1500 nm having a central wavelength of about 1300 nm. In an embodiment, RACAS radiates calibration light at a wavelength of about 1400 nm at which light is strongly absorbed by water and has an absorption coefficient approximately the same as that of light 2350.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE FIGURES

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature in a figure of an embodiment of the disclosure may be used to reference the given feature. Dimensions of features shown in the figures are chosen for convenience and clarity of presentation and are not necessarily shown to scale

FIGS. 1A and 1B schematically show perspective top and side views of a RACAS comprising canister light detectors mounted to motion stages, in accordance with an embodiment of the disclosure;

FIG. 1C shows a schematic cross section of RACAS shown in FIGS. 1A and 1B, in accordance with an embodiment of the disclosure;

FIG. 1D schematically shows a perspective view of a finger of a person's hand positioned in a finger cradle of RACAS shown in in FIGS. 1A-1C, in accordance with an embodiment of the disclosure;

FIG. 1E shows a schematic cross section of the finger in the cradle shown in FIG. 1D, in accordance with an embodiment of the disclosure;

FIGS. 2A and 2B schematically show cross section and perspective views respectively of a RACAS comprising a single canister light detector mounted to a finger cradle in a fixed position, in accordance with an embodiment of the disclosure;

FIG. 2C schematically shows a side cross section view of a finger positioned in RACAS shown in FIGS. 2A and 2B, in accordance with an embodiment of the disclosure; and

FIGS. 3A and 3B schematically show perspective and cross section views of a canister detector in accordance with an embodiment of the disclosure.

### DETAILED DESCRIPTION

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment in an application for which it is intended. Wherever a general term in the disclosure is illustrated by reference to an example instance or a list of example instances, the instance or instances referred to, are by way of non-limiting example instances of the general term, and the general term is not intended to be limited to the specific example instance or instances referred to. The phrase “in an embodiment”, whether or not associated with a permissive, such as “may”, “optionally”, or “by way of example”, is used to introduce for consideration an example, but not necessarily required, configuration of possible embodiments of the disclosure. Each of the verbs, “comprise” “include” and “have”, and conjugates thereof, are used to indicate that the object or objects of the verb are not necessarily a complete listing of components, elements or parts of the subject or subjects of the verb. Unless otherwise indicated, the word “or” in the description and claims is considered to be the inclusive “or” rather than the exclusive or, and indicates at least one of, or any combination of more than one of items it conjoins.

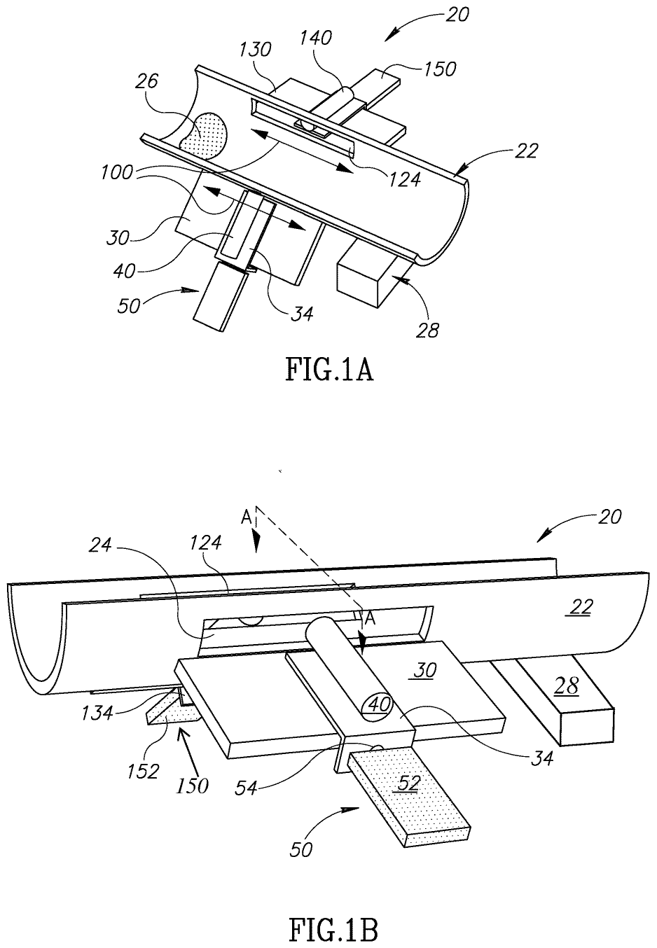

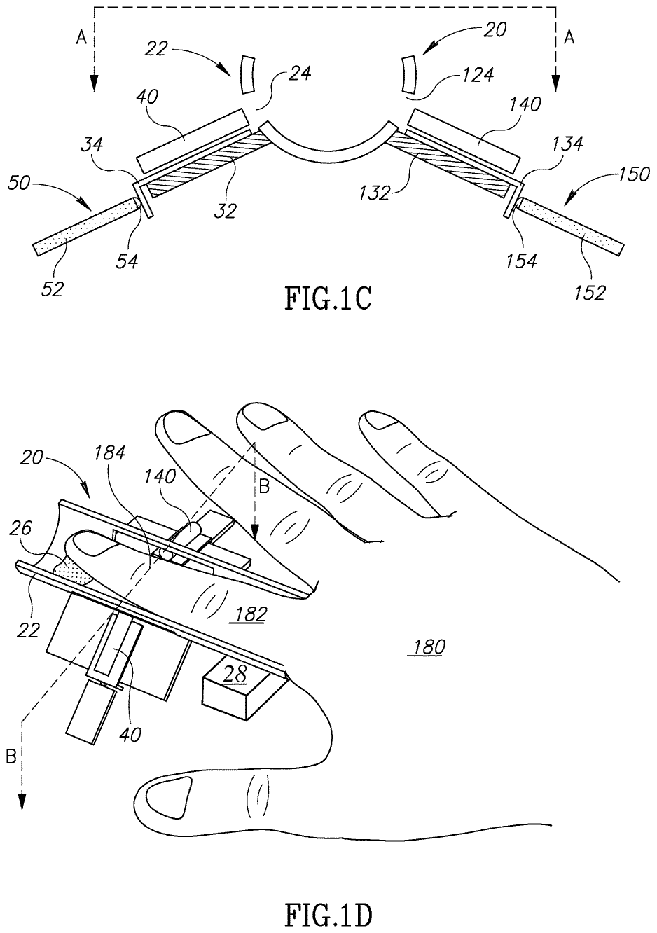

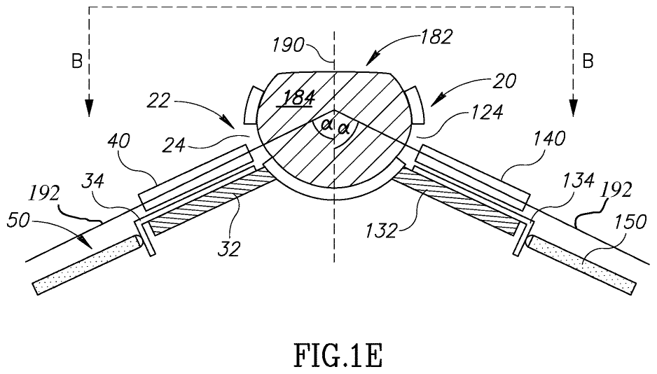

FIG. 1A schematically shows a top perspective view of a RACAS 20 in accordance with an embodiment of the disclosure. RACAS 20 optionally comprises a finger cradle 22 for receiving a finger of a person having a blood component assayed, a fingerprint detector 26 and canister detectors 40 and 140 that are respectively mounted to motion stages 34 and 134 on opposite sides of the cradle. Motion stages 34 and 134 are coupled to support fins 30 and 130 that are fixed to cradle 22 and are translatable along the support fins in directions indicated by double arrowhead arrows 100 optionally by piezoelectric motors 50 and 150. Piezoelectric motors 50 and 150 respectively comprise piezoelectric vibrators 52 and 152 that are friction coupled to their associated motion stages by friction nubs 54 and 154 (see FIGS. 1B and 1C).

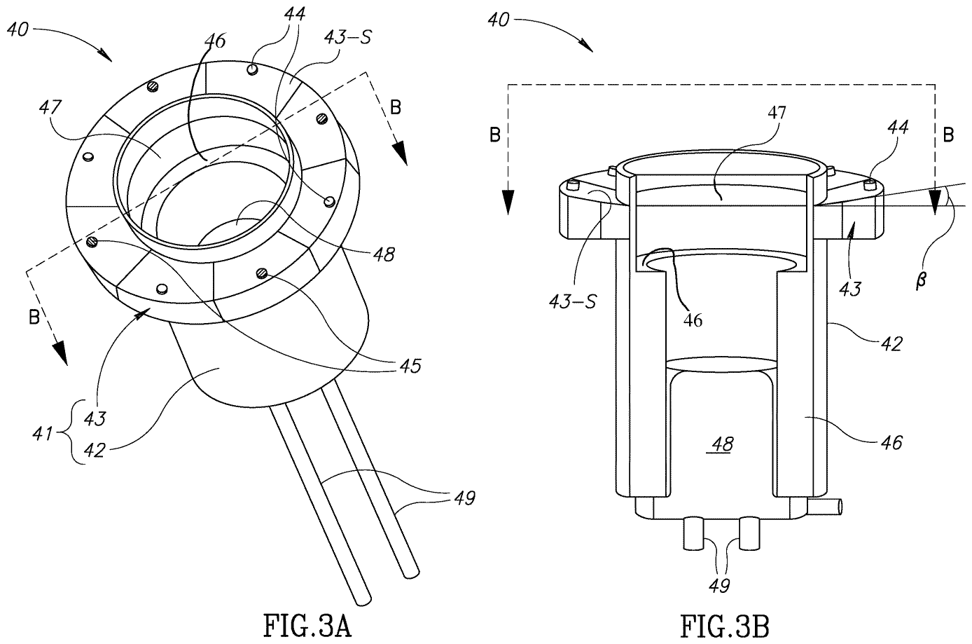

Finger cradle 22 is formed having apertures 24 and 124 of which only aperture 124 is seen in FIG. 1A. Aperture 24 is shown in FIGS. 1B and 1C. Optionally, each canister detector, as discussed in detail below with reference to FIGS. 3A and 3B, comprises at least one sensor for sensing assaying and/or calibration light, at least one light source operable to radiate assaying light, and at least one light source operable to radiate calibration light. The canister detectors are oriented on their respective motion stages to transmit and sense assaying and/or calibration light through apertures 24 and 124. A RACAS controller 28 controls canister detectors and motion stages.

FIG. 1B schematically shows an enlarged, side perspective view of RACAS 20 in which details of the configuration of canister detector 40, motion stage 34, support fin 30, and piezoelectric motor 50 are readily seen. Details of features of canister detector 140 and components of RACAS 20 associated with canister detector 140 are similar to details of canister detector 40 and to features associated with canister detector 40 shown in FIG. 1B.

FIG. 1C shows a schematic cross section view of RACAS 20 along a cross section plane A-A, also indicated for reference in FIG. 1B, in which canister detectors 40 and 140 and features associated with canister detectors 40 and 140 are shown.

In operation, a person who is to have a blood component assayed by RACAS 20 places a finger in cradle 22 with the fingertip positioned so that the finger's fingerprint is located on fingerprint detector 26 (FIG. 1A). Controller 28 processes signals from fingerprint detector 26 that the fingerprint detector generates responsive to placement of the fingertip on the detector to determine if a fingerprint is positioned on the fingerprint detector and optionally to identify a person to whom the fingerprint belongs. If controller 28 determines that there is a fingerprint on the fingerprint detector and, optionally, identifies the person, controller 28 controls piezoelectric motors 50 and 150 and canister detectors 40 and 140 to locate the DIPJ of the finger and position the canister detectors at operating positions substantially opposite the DIPJ.

In an embodiment to determine an operating position for canister detector 40 in a neighborhood substantially opposite the DIPJ, RACAS controller 28 controls piezoelectric motor 40 to move motion stage 34 and thereby position the canister detector at different locations along aperture 24. At each of the positions the controller controls the canister detector to radiate assaying light and/or calibration light through aperture 24 to illuminate the finger and sense intensity of assaying and/or calibration light that emerges from the finger responsive to the illumination. The controller processes the sensed light at the different positions to determine a position of the canister detector at which intensity of the sensed light exhibits a feature, for example a minimum or maximum, that indicates that the canister detector is located substantially opposite the DIPJ. Controller 28 determines an operating position for canister detector 40 based on the position at which the indicating feature is exhibited. In an embodiment an operating position at which to locate canister detector 140 is determined similarly to the manner in which an operating position is determined for canister detector 40.

In an embodiment, for determining location of the DIPJ and an operating position for canister detector 40 or canister detector 140, the canister detector may comprise at least one light source that radiates light in a bandwidth for which reflected light exhibits a relatively large change at the DIPJ. For example the light may be radiated in a bandwidth for which light is strongly reflected by bone.

Additionally or alternatively, to determine operating positions for canister detectors 40 and 140 RACAS controller may control detector 40 to radiate light towards the person's finger from different positions of the canister detector along aperture 24 and control canister detector 140 to sense light from canister detector 40 at different positions of canister detector 140 along aperture 124 that passes through the finger. A maximum in the sensed light may be used to indicate location of the DIPJ and suitable operating positions for the canister detectors.

FIG. 1D schematically shows a person's hand 180 and the hand's index finger 182 located in RACAS cradle 22 so that the tip of the finger and the finger's fingerprint (not shown) is located on fingerprint detector 26 and detector canisters 40 and 140 are located at operating positions opposite DIPJ 184 of finger 182.

FIG. 1E shows a schematic cross section view of finger 182 in cradle 22 of RACAS 20 at a cross section plane B-B also indicated in FIG. 1D. The figure shows a vertical cross section axis 190 of RACAS 20 in cross section plane B-B and pose angles & at which axes 192 of canister detectors 40 and 140 are oriented with respect to the cross section axis. Advantageously pose angles o are determined to enhance exposure of the proper digital arteries (not shown), distal transverse palmar arch and capillaries of finger 182 in the region of DIPJ 184. The proper digital arteries lie along the palmer side of the finger.

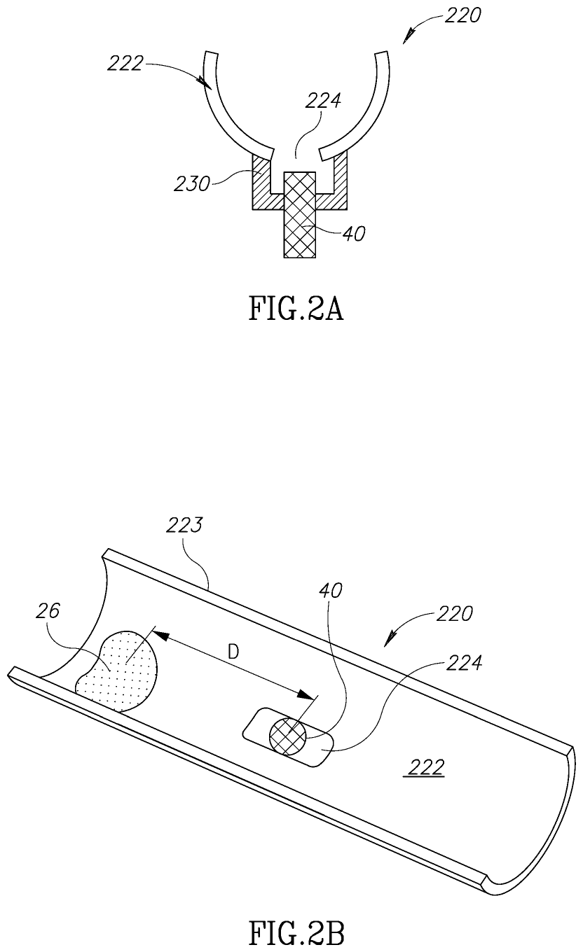

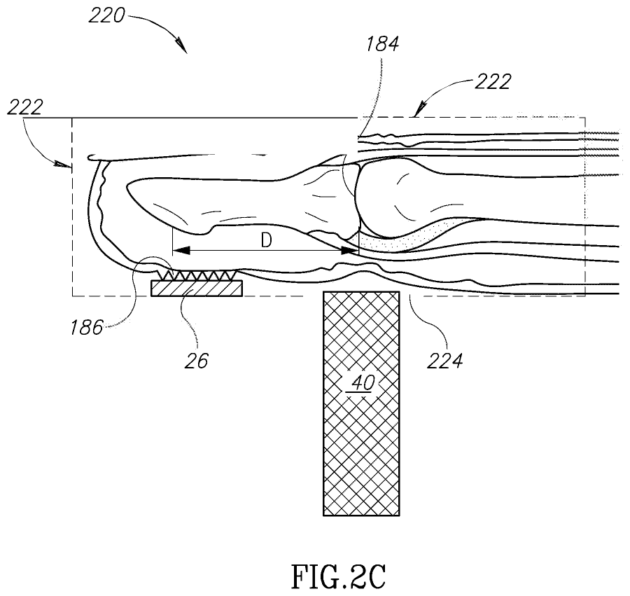

FIGS. 2A and 2B schematically show cross section and top perspective views respectively of a RACAS 220, in accordance with an embodiment of the disclosure. RACAS 220 optionally comprises a cradle 222 and a single canister detector 40 mounted to the cradle and operable to radiate assaying and calibration light towards a finger (not shown) in the cradle through an aperture 224 formed in the cradle. Canister detector 40 is optionally mounted, as indicated in FIG. 2A, in a fixed position relative to aperture 224 by a support bracket 230. In an embodiment as schematically shown in FIG. 2B, RACAS 220 comprises a fingerprint detector 26 and canister detector 40 is located at a fixed distance D from the fingerprint detector. Advantageously, distance D is equal to an average distance at which the DIPJ of an, optionally index, finger is located relative to the index finger fingerprint. Optionally, fingerprint detector determines the location of a fingerprint based on a location of a central pocket whorl or loop of the fingerprint.

In an embodiment, single canister detector 40 in a RACAS 220 may not be located at a fixed position. The canister detector may be mounted to a motion stage (not shown) so that the canister detector may be translated along aperture 224 and controlled, optionally as described above with respect to motion stages 34 and 134, to locate the DIPJ of a finger and position canister detector 40 at an operating position based on the DIPJ location.

FIG. 2C schematically shows a side cross section view of a finger 182 positioned in cradle 222 for assaying a blood component by RACAS 220, in accordance with an embodiment of the disclosure. A fingerprint 186 of finger 182 is properly positioned on fingerprint detector 26 and canister detector 40 is located at a suitable operating position below DIPJ 184 of the finger at a distant D from the fingerprint, in accordance with an embodiment of the disclosure.

FIGS. 3A and 3B schematically show perspective and cutaway views of a canister detector 40 in accordance with an embodiment of the disclosure.

Canister detector 40 as schematically shown in FIG. 3A optionally comprises a canister housing 41 comprising a can 42 having a rim 43. A plurality of light sources 44 that are controllable to radiate assaying light are arrayed on a surface 43-S of rim 43. Light sources 44 are, optionally interleaved, on surface 43-S with a plurality of light sources 45, shown shaded, that are controllable to radiate calibration light. Light sources 44 and 45 may be light emitting diodes (LEDs) or laser diodes. A light sensor 48, such as a photodiode, that generates signals responsive to intensity of assaying and calibration light incident on the detector is seated below rim 43 inside a collimating tube 46 in can 42. The canister detector optionally comprises a collecting lens 47 configured to direct light incident on the lens to light sensor 48. Control leads represented by leads 49 are useable to connect canister detector 40 to a controller, such as controller 28 shown in FIG. 1A, operable to control light sources 44, 45, and light sensor 48 and receive signals that the detector generates responsive to incident light.

In an embodiment surface 43-S is a curved surface of a conical frustum having a tilt angle β as shown in the cutaway perspective view of cannister detector 40 in FIG. 3B. Magnitude of tilt angle β is determined so that beams of assaying and calibration light that light sources 44 and 45 radiate overlap at a distance from rim 43 that is suitable for illuminating a region of a DIPJ advantageous for assaying a blood component in accordance with an embodiment of the disclosure.

In an embodiment RACAS 20 or 220 operates to provide a measure of BAC. Light sources 44 may be LEDs that are controllable to radiate assaying light in a bandwidth between about 2,000 nm and 2500 nm having a central wavelength of about 2350 nm that is strongly absorbed by ethanol. Light sources 45 may comprise LEDs that radiate calibration light in a bandwidth between about 1100 and 1500 nm having a central wavelength of about 1300 nm that is substantially not absorbed by ethanol and/or in a bandwidth having a central wavelength of about 1400 nm at which light is strongly absorbed by water.

It is noted that whereas in the discussion above a finger cradle is schematically shown as a cradle that is a portion of a circular cylinder, embodiments of a RACAS are not limited to cradles that are portions of a circular cylinder. A RACAS cradle may for example be planar or be a cradle that completely encloses a finger.

It is further noted that whereas in the above description the DIPJ of a finger is indicated as an assay region of the finger, practice of an embodiment of the disclosure is not limited to assay regions that are DIPJs. For example, an assay region of finger 390 may be the pulp of the fingertip of the finger.

Whereas the specification describes an embodiment of a RACAS as comprising canister light detectors comprising both light sources and a light sensor, a RACAS may comprise light sources and light sensors as separate devices. For example, a RACAS in accordance with an embodiment may have at least one light source that radiates light to illuminate DIPJ 184 shown in FIG. 1E from an optionally fixed pose with light in a field of illumination (FOI) having a central axis oriented at about 40° relative to axis 190 shown in the figure. The RACAS may have at least one light sensor for receiving light from the DIPJ in a field of view (FOV) having a central axis located at about 40° on a side of axis 190 opposite to that of the at least one light source.

Apparatus for assaying a blood component of a person, the apparatus comprising: a cradle for receiving an assay region of person's finger; at least one first light source operable to illuminate the assay region when received by the cradle with assaying light that is relatively strongly absorbed by the blood component; at least one second light source operable to illuminate the assay region with calibration light that is relatively weakly or substantially not absorbed by the blood component; at least one light sensor that generates signals responsive to assaying and calibration light that emerges from the finger responsive to illumination by light from the at least one first and the at least one second light source; and a controller that processes signals from the at least one light sensor to determine an assay of the blood component; and a fingerprint detector operable to generate a fingerprint signal indicating presence of a fingerprint of a finger received by the cradle; wherein the controller provides an assay of the blood component responsive to receiving the fingerprint signal.

Optionally, the at least one fingerprint detector is located at fixed distances relative to locations of the at least one first light source, the at least one second light source, and the at least one light sensor. Optionally, the at least one first and/or at least one second light source are moveable. Optionally, the controller is operable to move and align the at least one first and/or second light source so that light from the at least one first and/or second light source is incident on the assay region.

In an embodiment, the at least one light sensor is moveable. Optionally, the controller is operable to move the at least one light sensor to align the at least one light source with the assay region so that light that emerges from the finger responsive to illumination by light from the at least one first and the at least one second light source is incident on the at least one light detector.

In an embodiment, the at least one light source and at least one light sensor are comprised in a same housing. Optionally, the housing comprises a can having first and second ends and an external rim at a first end of the can; a plurality of light sources located on the rim and oriented so that light radiated by the plurality of light sources is directed to overlap in a same focal region; and at least one light sensor located inside the can to receive light from the focal region. Optionally, the at least one light sensor is displaced from the plurality of light sources towards the second end of the can.

In an embodiment, the processor is configured to determine if the assay of the blood component is aberrant. In an embodiment, the fingerprint signal comprises an image of the fingerprint. Optionally, the controller is configured to process the fingerprint image to determine an identity of a person whose finger is received by the cradle. Optionally, the processor uses the identity to determine if the assay is aberrant. Additionally or alternatively, the processor may determine that the assay is aberrant if the assay is outside of a desired range.

In an embodiment the assay region is a region comprising a distal phalangeal joint (DIPJ) of the person's finger. In an embodiment the blood component is ethyl alcohol.

There is further provided in accordance with an embodiment a light detector comprising: a housing comprising a can having first and second ends and an external rim at a first end of the can; a plurality of light sources located on the rim and oriented so that light radiated by the light sources is directed to overlap in a same focal region; and at least one light sensor located inside the can to receive light from the focal region. Optionally, the at least one light sensor is displaced from the plurality of light sources towards the second end of the can.

There is further provided in accordance with an embodiment of the disclosure a method for assaying a blood component of a person, the method comprising: illuminating a distal phalangeal joint (DIPJ) of the finger with assaying light that is strongly absorbed by the blood component; illuminating the DIPJ with calibration light that is relatively weakly or substantially not absorbed by the blood component and with calibration light that is strongly absorbed by water; detecting assaying and calibration light emergent from the DIPJ responsive to illumination by the assaying and calibration light; and processing the detected assaying and calibration light to assay the blood component. Optionally, the blood component is ethyl alcohol.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims

1. Apparatus for assaying a blood component of a person, the apparatus comprising:
a cradle for receiving an assay region of person's finger;
at least one first light source operable to illuminate the assay region when received by the cradle with assaying light that is relatively strongly absorbed by the blood component;
at least one second light source operable to illuminate the assay region with calibration light that is relatively weakly or substantially not absorbed by the blood component;
at least one light sensor that generates signals responsive to assaying and calibration light that emerges from the finger responsive to illumination by light from the at least one first and the at least one second light source; and
a controller that processes signals from the at least one light sensor to determine an assay of the blood component; and
a fingerprint detector operable to generate a fingerprint signal indicating presence of a fingerprint of a finger received by the cradle;
wherein the controller provides an assay of the blood component responsive to receiving the fingerprint signal.

2. The apparatus according to claim 1 wherein the at least one fingerprint detector is located at fixed distances relative to locations of the at least one first light source, the at least one second light source, and the at least one light sensor.

3. The apparatus according to claim 1 wherein the at least one first and/or the at least one second light source are moveable.

4. The apparatus according to claim 3 wherein the controller is operable to move and align the at least one first and/or second light source so that light from the at least one first and/or second light source is incident on the assay region.

5. The apparatus according to claim 1 wherein the at least one light sensor is moveable.

6. The apparatus according to claim 5 wherein the controller is operable to move the at least one light sensor to align the at least one light source with the assay region so that light that emerges from the finger responsive to illumination by light from the at least one first and the at least one second light source is incident on the at least one light detector.

7. The apparatus according to claim 1 wherein the at least one light source and at least one light sensor are comprised in a same housing.

8. The apparatus according to claim 7 wherein the housing comprises a can having first and second ends and an external rim at a first end of the can;
a plurality of light sources located on the rim and oriented so that light radiated by the plurality of light sources is directed to overlap in a same focal region; and
at least one light sensor located inside the can to receive light from the focal region.

9. The apparatus according to claim 8 wherein the at least one light sensor is displaced from the plurality of light sources towards the second end of the can.

10. The apparatus according to claim 1 wherein the processor is configured to determine if the assay of the blood component is aberrant.

11. The apparatus according to claim 1 wherein the fingerprint signal comprises an image of the fingerprint.

12. The apparatus according to claim 11 wherein the controller is configured to process the fingerprint image to determine an identity of a person whose finger is received by the cradle.

13. The apparatus according to claim 12 wherein the processor uses the identity to determine if the assay is aberrant.

14. The apparatus according to claim 10 wherein the processor determines that the assay is aberrant if the assay is outside of a desired range.

15. The apparatus according to claim 1 wherein the assay region is a region comprising a distal phalangeal joint (DIPJ) of the person's finger.

16. The apparatus according to claim 1 wherein the blood component is ethyl alcohol.

17. A light detector comprising:
a housing comprising a can having first and second ends and an external rim at a first end of the can;
a plurality of light sources located on the rim and oriented so that light radiated by the light sources is directed to overlap in a same focal region; and
at least one light sensor located inside the can to receive light from the focal region.

18. The light detector according to claim 17 wherein the at least one light sensor is displaced from the plurality of light sources towards the second end of the can.

19. A method for assaying a blood component of a person, the method comprising:
illuminating a distal phalangeal joint (DIPJ) of the finger with assaying light that is strongly absorbed by the blood component;
illuminating the DIPJ with calibration light that is relatively weakly or substantially not absorbed by the blood component and with calibration light that is strongly absorbed by water;
detecting assaying and calibration light emergent from the DIPJ responsive to illumination by the assaying and calibration light; and
processing the detected assaying and calibration light to assay the blood component.

20. The apparatus according to claim 19 wherein the blood component is ethyl alcohol.
